(19) United States
(12) Patent Application Publication
(10) Pub. No.: US 2021/0045785 A1
(43) Pub. Date: Feb. 18, 2021

(54) POLYAXIAL FIXATION SYSTEM FOR REVISION SURGERY

(71) Applicant: Premia Spine Ltd., Ramat Poleg (IL)

(72) Inventor: Rafi Haziza, Kiryat Bialik (IL)

(73) Assignee: Premia Spine Ltd., Ramat Poleg (IL)

(21) Appl. No.: 16/541,274

(22) Filed: Aug. 15, 2019

(51) Int. Cl. A61B 17/70; A61F 2/46

(52) U.S. Cl. CPC A61B 17/7082; A61F 2002/3069; A61B 17/7008; A61F 2/4611

(57) ABSTRACT

A spinal system includes a polyaxial fastener including a setscrew and a nut. The setscrew includes an externally-threaded base, which is formed with an inner spherical race, and a polyaxial setscrew member, which includes a spherical base that extends from an externally-threaded setscrew head. The spherical base is received in the inner spherical race.

### FIELD OF THE INVENTION

The present invention relates generally to methods and apparatus for minimally invasive surgery on spinal structures, and particularly to a polyaxial fixation system, such as for spinal fixation revision surgery.

### BACKGROUND OF THE INVENTION

Spinal fixation systems are known for treating patients suffering from lumbar spinal disease that requires decompression and stabilization by fixation.

In some spinal fixation surgical techniques, a spinal rod connects screws inserted into adjacent vertebral bodies in order to prevent motion and allow fusion to occur across adjacent spine segments. After spinal fusion operation, sometimes a patient can develop problems at the spinal levels above or below the fusion, necessitating another surgery, called revision surgery, which involves extension of the previously implanted devices.

In revision surgery, the existing implants may be removed and replaced with new implants. Alternatively, devices may be linked to the existing devices, which may decrease the surgical morbidity for the patient, because the previously placed devices are maintained in place.

### SUMMARY OF THE INVENTION

The present invention seeks to provide a polyaxial fixation system, such as for spinal fixation revision surgery, as described in more detail further below.

There is thus provided in accordance with a non-limiting embodiment of the present invention a spinal system including a polyaxial fastener including a setscrew and a nut, the setscrew including an externally-threaded base, which is formed with an inner spherical race, and a polyaxial setscrew member, which includes a spherical base that extends from an externally-threaded setscrew head, wherein the spherical base is received in the inner spherical race.

In one embodiment, the setscrew head includes a first wrench socket and the threaded base includes a second wrench socket.

In one embodiment, the nut includes a threaded aperture and a lower concave surface, and the threaded aperture mates with the externally-threaded setscrew head.

In one embodiment, the system includes an implant, which has an annular portion from which extends a rod, and the annular portion has a through hole which has a concave inner contour and a convex outer contour whose curvature matches that of the lower concave surface of the nut. The polyaxial nut is screwed on the setscrew head of the polyaxial setscrew member and tightens the annular portion of the implant against a tulip head of a screw at a desired rotational orientation, thereby fixing the polyaxial setscrew member at the desired rotational orientation with respect to the threaded base.

In one embodiment, the rod is coupled to the annular portion by means of a polyaxial joint, which is lockable with a locking element.

In one embodiment, an implant installation measurement tool is configured to measure implant parameters, including at least one of up/down offsets, medial/lateral offsets, and implant angular orientation.

In one embodiment, the implant installation measurement tool includes a first measurement member and a second measurement member, and wherein a first offset gauge extends outwards from the first measurement member and a pointer is coupled to the second measurement member and points to markings on the first offset gauge.

In one embodiment, the implant installation measurement tool further includes a second offset gauge for measuring an offset different from the first offset gauge.

In one embodiment, the implant installation measurement tool further includes an implant angle gauge configured to measure an angular orientation of the first measurement member with respect to the second measurement member.

In one embodiment, the spinal system further includes a cannula guide, which includes a hollow body formed with a longitudinal slot that extends from a head portion to an end portion.

### BRIEF DESCRIPTION OF THE DRAWINGS

The present invention will be understood and appreciated more fully from the following detailed description, taken in conjunction with the drawings in which:

FIG. 1 is a simplified illustration of a cannula guide 10, constructed and operative in accordance with a non-limiting embodiment of the present invention.

FIGS. 2A-2D are simplified illustrations of using the cannula guide to remove an existing screw, such as in the beginning of revision surgery, in accordance with a non-limiting embodiment of the present invention.

FIGS. 3A, 3B, 3C and 3D are simplified illustrations of an implant installation measurement tool, constructed and operative in accordance with a non-limiting embodiment of the present invention.

FIG. 4 is a simplified illustration of a demo implant, which may serve as a template for measuring implant parameters, in accordance with a non-limiting embodiment of the present invention.

FIGS. 5 and 5A-5D are simplified illustrations of an implant, which may be used in a revision procedure, in accordance with a non-limiting embodiment of the invention.

FIG. 6 is a simplified illustration of installing the implant in a revision procedure, in accordance with a non-limiting embodiment of the present invention.

FIGS. 7A, 7B and 7C are simplified illustrations of securing a rod of the implant to a head of a fastener in the revision procedure, in accordance with a non-limiting embodiment of the present invention.

FIGS. 8A-8C are simplified illustrations of a setscrew of a polyaxial fastener, in accordance with a non-limiting embodiment of the present invention.

FIGS. 8D-8E are simplified illustrations of a nut of a polyaxial fastener, in accordance with a non-limiting embodiment of the present invention.

FIGS. 9A-9C are simplified illustrations of an implant, which may be used in a revision procedure with a curved spinal rod, in accordance with a non-limiting embodiment of the invention.

### DETAILED DESCRIPTION OF EMBODIMENTS

Reference is now made to FIG. 1, which illustrates a cannula guide 10, constructed and operative in accordance with a non-limiting embodiment of the present invention.

Cannula guide 10 includes a cylindrical hollow body 11 formed with a longitudinal slot 12 that extends from a head portion 14 (optionally flared-out) to an end portion 16. Body 11 may be formed with external threads or helical grooves 18. Body 11 may be rigid, but in a preferred embodiment it is flexible and resilient to give a wider angular range of working possibilities for the surgeon. Accordingly, body 11 may be made of stainless steel, plastic, elastomeric materials and others.

Cannula guide 10 has multiple uses, such as but not limited to, a working tunnel for bone removal, cleaning tissue, and other actions; a counter force/torque member when opening and removing old screws and tightening new screws, nuts or other fasteners; a guide for inserting rods or other devices via the fully open slot 12, and other uses.

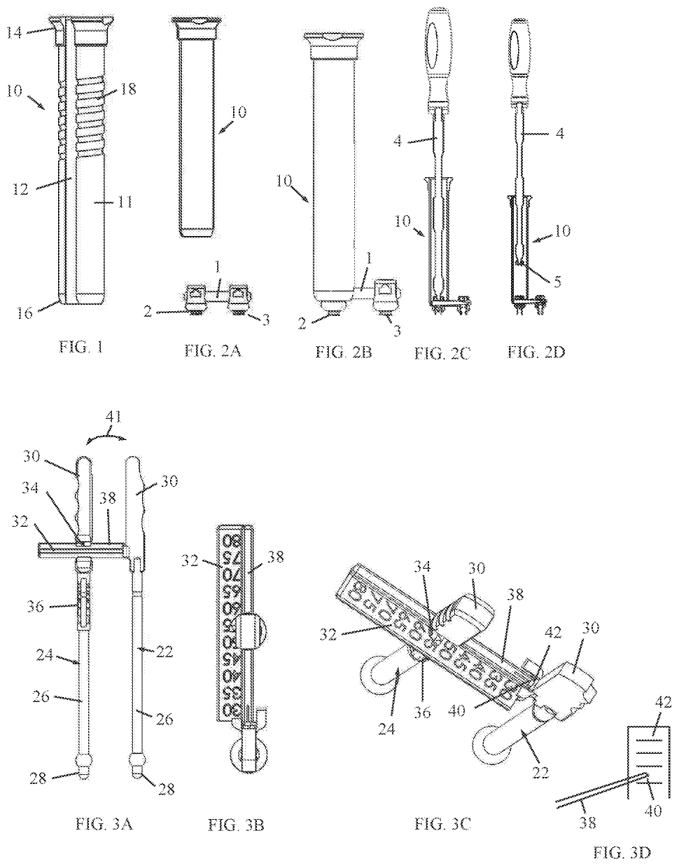

Reference is now made to FIGS. 2A-2D, which illustrate using the cannula guide 10 to remove an existing screw, such as in the beginning of revision surgery, in accordance with a non-limiting embodiment of the present invention.

In FIG. 2A, a previously implanted spinal rod 1 is affixed to two pedicle screws 2 and 3. In FIG. 2B, cannula guide 10 is placed over screw 2, which is the side at which a new extension will be attached, as described below. In FIG. 2C, a screwdriver 4 is introduced through and guided by cannula guide 10 to access screw 2. In FIG. 2D, screwdriver 4 has been used to remove a fastener 5 (e.g., setscrew or nut) from the head of pedicle screw 2.

Reference is now made to FIGS. 3A, 3B and 3C, which illustrate an implant installation measurement tool 20, constructed and operative in accordance with a non-limiting embodiment of the present invention. The measurement tool 20 is designed to measure all implant parameters (e.g., length, up/down, medial/lateral offsets, implant angular orientation) to help correctly select the new implant to be inserted in the revision surgery.

The implant installation measurement tool 20 includes a first measurement member 22 and a second measurement member 24. Each measurement member 22 and 24 includes a shaft 26 with a fastener interface 28 at one end thereof (for contacting an existing fastener) and a handle 30 at an opposite end thereof. A first offset gauge 32 (which may measure caudal/cephalad or medial/lateral offset) extends outwards (sideways) from first measurement member 22 and has markings for easily reading the amount of medial/lateral offset. A pointer 34 is coupled to second measurement member 24 and overlies and points to the markings on the first offset gauge 32.

One of the measurement members (such as second measurement member 24) is equipped with a second offset gauge 36 (e.g., an up/down gauge) which has suitable markings for easily reading the amount of up/down offset (or any other direction different from the first offset gauge 32). Each handle 30 is movable longitudinally on its shaft 26, either by threaded movement or sliding movement, for example, in order to arrive at the desired amount of up/down offset.

An implant angle gauge may be provided, such as a bar 38 that extends along or is part of medial/lateral offset gauge 32. As seen best in FIG. 3D, the end of the bar 38 nearest first measurement member 22 has a pointer 40 that points to an angular measurement scale 42 affixed to first measurement member 22. This measures the angular orientation of first measurement member 22 with respect to second measurement member 24, as indicated by arrow 41 in FIG. 3A.

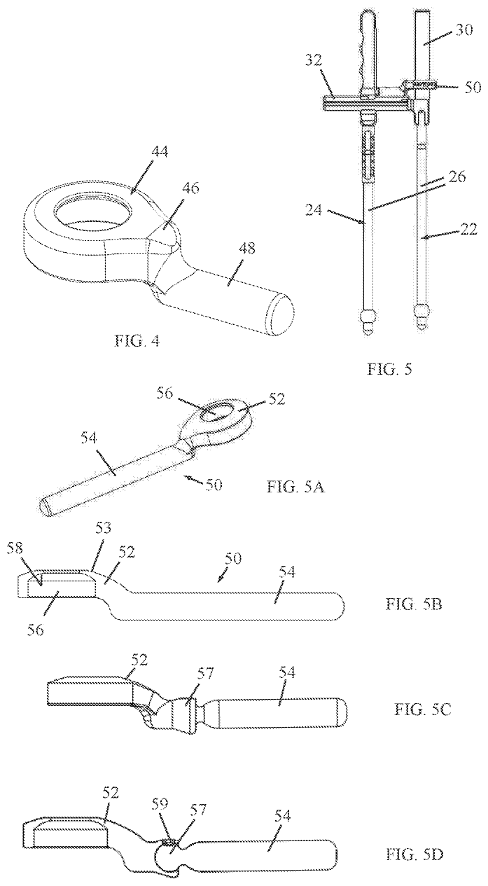

Accordingly, the measurement tool 20 can measure all the necessary implant parameters. The invention provides alternatives for making these measurements. Reference is now made to FIG. 4, which illustrates a demo implant 44, which may serve as a template for measuring implant parameters, in accordance with a non-limiting embodiment of the present invention.

Demo implant 44 may be made of a flexible material (e.g., elastomeric) and placed in the implantation site in the patient's body. Demo implant 44 has a flexible annular portion 46 from which extends a flexible and impressionable rod 48. After placing the demo implant 44 in the implantation site in the patient's body, a setscrew (not shown) may be tightened against the flexible and impressionable rod 48. The setscrew will leave a mark on rod 48; this mark will be used to measure the actual length of rod required in the real implant. The flexible elements 46 and 48 adjust to the location needed for the implant.

Reference is now made to FIGS. 5 and 5A-5B, which illustrate an implant 50, which is the real implant meant for the revision procedure. As seen in FIGS. 5A-5B, implant 50 has an annular portion 52 from which extends a rod 54. The annular portion 52 has a through hole 56 which has a concave (e.g., domed, spherical or conical) inner contour 58 and a convex outer contour 53.

As seen in FIG. 5, implant 50 may be simply placed on first measurement member 22, with shaft 26 or handle 30 going through the hole of the implant. All the adjustments and measurements of the measurement members 22 and 24 are made with the real implant 50 in place, so that all the implant parameters are correctly established because implant 50 replicates its position in the body via the measurement members 22 and 24.

Another version of implant 50 is shown in FIGS. 5C and 5D. In this version, rod 54 is coupled to annular portion 52 by means of a polyaxial joint 57, such as a ball and socket joint that provides multiple angular orientation of rod 54 with respect to annular portion 52. After setting the desired angular orientation of rod 54 with respect to annular portion 52, the joint 57 may be locked with a locking element, such as a screw 59.

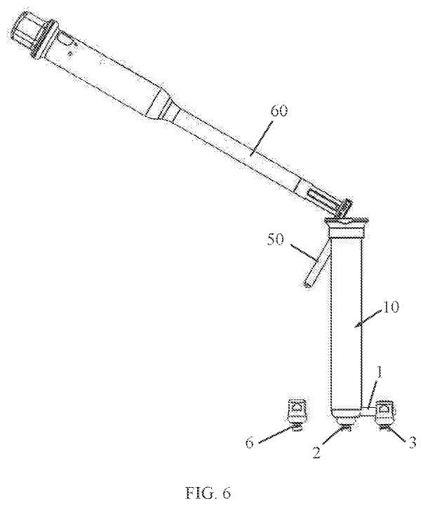

Reference is now made to FIG. 6, which illustrates installing the implant 50 in a revision procedure, in accordance with a non-limiting embodiment of the present invention. Implant 50 may be grasped by a grasping tool 60, and introduced in the slot of cannula guide 10. One of the objects of the revision procedure is to install the annular portion of the implant 50 on the existing screw 2 and the rod of the implant 50 on another screw 6, which is offset from screw 2.

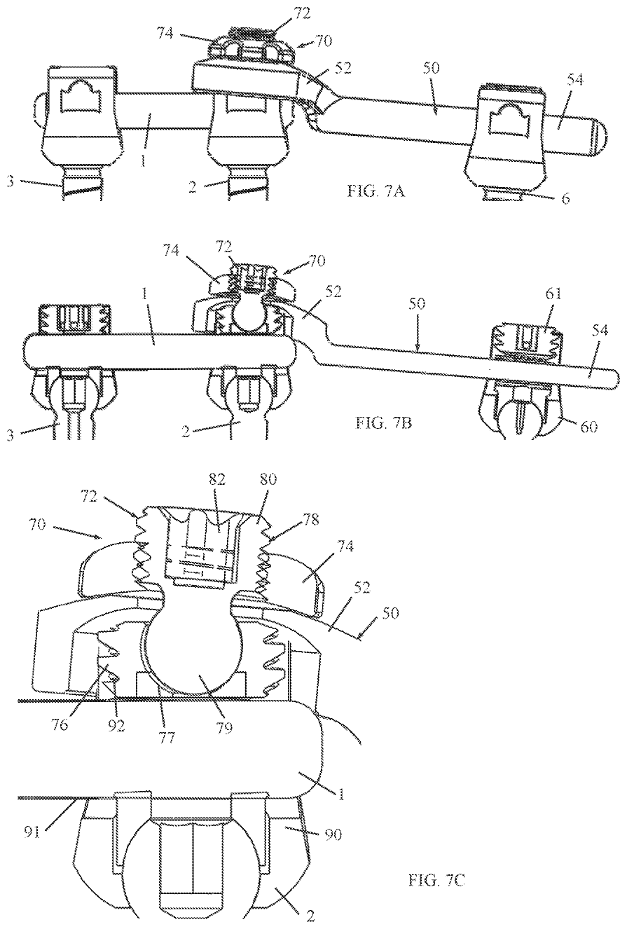

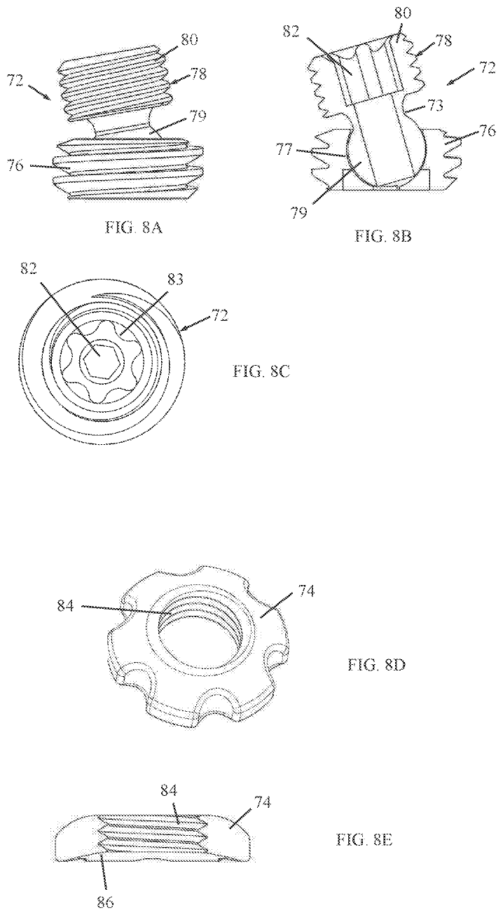

Referring to FIGS. 7A and 7B, rod 54 is secured to the “tulip” head of screw 6, such as by a setscrew or nut 61 (FIG. 7B). The annular portion 52 of implant 50 may be secured to the head of screw 2, such as by means of a novel polyaxial fastener 70, which is now described with reference to FIGS. 7C and 8A-8E. The polyaxial fastener 70 includes a modified setscrew 72 (FIGS. 8A-8C) and a modified nut 74 (FIGS. 8D-8E). The setscrew 72 includes an externally-threaded base 76, which is formed with an inner spherical race 77, and a polyaxial setscrew member 78, which includes a spherical base 79 that extends from an externally-threaded setscrew head 80, such as at a narrow neck junction 73. The spherical base 79 is received in inner spherical race 77. The setscrew head 80 may have a wrench socket 82, such as a hexagonal Allen-wrench head 82, as seen in FIG. 8C. A screwdriver (not shown) can be inserted in the wrench socket 82 to provide a counter torque when tightening nut 74 to secure the annular portion 52 on the top of the tulip of screw 2.

As seen in FIG. 8C, the externally-threaded base 76 may have another wrench socket 83, such as a Torx socket. In this manner a (Torx) screwdriver (not shown) can be used to tighten threaded base 76 against rod 1.

As seen in FIGS. 8D-8E, nut 74 may include a central threaded aperture 84 and a lower concave (e.g., spherical) surface 86, which matches the curvature of convex outer contour 53 (FIG. 5B). The internally-threaded aperture 84 mates with the externally-threaded setscrew head 80.

FIG. 7C shows the finished assembly of the revision procedure. The annular portion 52 of implant 50 has been placed over the “tulip” head 90 of screw 2. Rod 1 is secured in an aperture 91 of tulip head 90. The upper part of tulip head 90 has an internal thread 92 which mates with the externally-threaded base 76. Accordingly, as mentioned before, a (Torx) screwdriver (not shown) can be inserted in wrench socket 83 to tighten threaded base 76 against rod 1. The polyaxial nut 74 is screwed on setscrew head 80 of the polyaxial setscrew member 78 and tightens the annular portion 52 of implant 50 against the tulip head 90 of screw 2 at a desired rotational orientation. The polyaxial setscrew member 78 is thus fixed at this desired rotational orientation with respect to base 76.

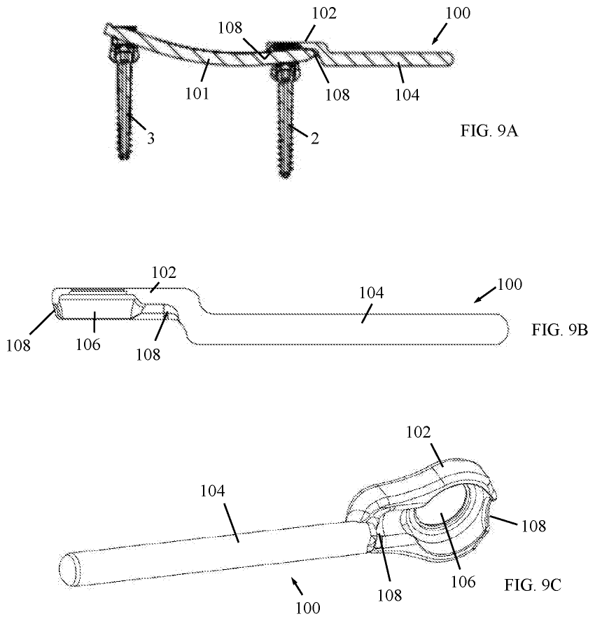

Reference is now made to FIGS. 9A-9C, which illustrate an implant 100, which may be used in a revision procedure with a curved spinal rod 101, in accordance with a non-limiting embodiment of the invention. Similar to implant 50 of FIG. 5, implant 100 has an annular portion 102 from which extends a rod 104. The annular portion 102 has a through hole 106 for accepting a setscrew as in other embodiments. The underside of annular portion 102 (the side facing the pedicle screw) is formed with one or more curved cutouts 108 which receive therein portions of the curved spinal rod 101. As can be seen in FIG. 9A, the cutouts 108 accommodate the curved structure of rod 101.

What is claimed is:

1. A spinal system comprising:
a polyaxial fastener comprising a setscrew and a nut, said setscrew comprising an externally-threaded base, which is formed with an inner spherical race, and a polyaxial setscrew member, which comprises a spherical base that extends from an externally-threaded setscrew head, wherein said spherical base is received in said inner spherical race.

2. The spinal system according to claim 1, wherein said setscrew head comprises a first wrench socket and said threaded base comprises a second wrench socket.

3. The spinal system according to claim 1, wherein said nut comprises a threaded aperture and a lower concave surface, and said threaded aperture mates with said externally-threaded setscrew head.

4. The spinal system according to claim 4, further comprising an implant, which has an annular portion from which extends a rod, and wherein said annular portion has a through hole which has a concave inner contour and a convex outer contour whose curvature matches that of said lower concave surface.

5. The spinal system according to claim 4, wherein said polyaxial nut is screwed on said setscrew head of said polyaxial setscrew member and tightens said annular portion against a tulip head of a screw at a desired rotational orientation, thereby fixing said polyaxial setscrew member at said desired rotational orientation with respect to said threaded base.

6. The spinal system according to claim 4, wherein said rod is coupled to said annular portion by means of a polyaxial joint, which is lockable with a locking element.

7. The spinal system according to claim 1, further comprising an implant installation measurement tool configured to measure implant parameters, including at least one of up/down offsets, medial/lateral offsets, caudal/cephalad offsets, and implant angular orientation.

8. The spinal system according to claim 7, wherein said implant installation measurement tool comprises a first measurement member and a second measurement member, and wherein a first offset gauge extends outwards from said first measurement member and a pointer is coupled to said second measurement member and points to markings on said first offset gauge.

9. The spinal system according to claim 8, wherein said implant installation measurement tool further comprises a second offset gauge for measuring an offset different from the first offset gauge.

10. The spinal system according to claim 8, wherein said implant installation measurement tool further comprises an implant angle gauge configured to measure an angular orientation of said first measurement member with respect to said second measurement member.

11. The spinal system according to claim 1, further comprising a cannula guide, which comprises a hollow body formed with a longitudinal slot that extends from a head portion to an end portion.

12. The spinal system according to claim 1, further comprising an implant, which has an annular portion from which extends a rod, and wherein an underside of said annular portion is formed with one or more curved cutouts.
